(19) United States
(12) Patent Application Publication
(10) Pub. No.: US 2009/0035829 A1
(43) Pub. Date: Feb. 5, 2009

(54) Enzymatic process for the preparation of paliperidone and its intermediate CMHTP

(76) Inventors: Santiago Ini, Haifa (IL); Laszlo Toth, Balmazujvaros (HU); Lorant Szabo, Godollo (HU); Ronen Tchelet, Pardesia (IL)

Correspondence Address: KENYON & KENYON LLP, ONE BROADWAY, NEW YORK, NY, 10004, US

(21) Appl. No.: 12/154,437

(22) Filed: May 21, 2008

Related U.S. Application Data
(60) Provisional application No. 60/931,295, filed on May 21, 2007.

(51) Int. Cl. C12P 17/16

(52) U.S. Cl. 435/118

(57) ABSTRACT

The present invention provides a process for the preparation of a 4H-pyrrido[1,2-a]-pyrimidin-4-one derivative, wherein the 4H-pyrrido[1,2-a]-pyrimidin-4-one derivative is Paliperidone or 3-(2-chloroethyl)-6,7,8,9-tetrahydro-9-hydroxy-2-methyl-4H-pyrrido[1,2-a]-pyrimidin-4-one (‘CMHTP’), said process comprising enzymatically hydroxylating Risperidone or 3-(2-chloroethyl)-6,7,8,9-tetrahydro-2-methyl-4H-pyrrido[1,2-a]-pyrimidin-4-one (‘ClMTTP’), respectively, with at least one oxidoreductase enzyme; and optionally isolating or purifying the Paliperidone or CMHTP, wherein the at least one oxidoreductase enzyme is selected from the group of peroxidases, dioxygenases, monooxygenases and any combination thereof.

### CROSS REFERENCE TO RELATED APPLICATION

This application claims the benefit of U.S. Provisional Application No. 60/931,295 filed May 21, 2007, the disclosure of which is hereby incorporated by reference.

### FIELD OF THE INVENTION

The present invention is related to a process for the preparation of Paliperidone and 3-{2-[4-(6-fluoro-1,2-benzisoxazol-3-yl)-1-piperidinyl]ethyl}-2-methyl-4H-pyrrido[1,2-a]pyrimidin-4-one via enzymatic hydroxylation.

### BACKGROUND

Paliperidone, 3-[2-[4-(6-fluorobenzo[d]isoxazol-3-yl)-1-piperidyl]ethyl]-7-hydroxy-4-methyl-1,5-diazabicyclo[4.4.0]deca-3,5-dien-2-one or 3-{2-[4-(6-fluoro-1,2-benzisoxazol-3-yl)-1-piperidinyl]ethyl}-9-hydroxy-2-methyl-4H-pyrrido[1,2-a]pyrimidin-4-one, is a 5-HT antagonist belonging to the chemical class of benzisoxazole derivatives and a racemic mixture having the following structural formula:

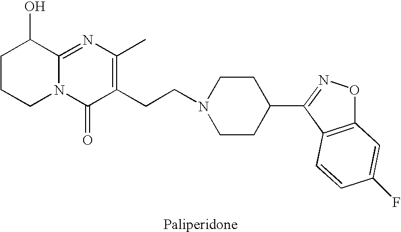

Paliperidone is a metabolite of Risperidone, 3-{2-[4-(6-fluoro-1,2-benzisoxazol-3-yl)-1-piperidinyl]ethyl}-2-methyl-4H-pyrrido[1,2-a]pyrimidin-4-one. Marketed under the name, Invega®, Paliperidone is a psychotropic agent approved in the United States for the treatment of schizophrenia.

Paliperidone is disclosed in U.S. Pat. Nos. 5,158,952, 5,254,556 and 6,320,048. WO 2006/114384 discloses a crystalline palmitate ester of Paliperidone. Other derivatives of Paliperidone are disclosed in U.S. Pat. No. 5,688,799.

A process for the synthesis of Paliperidone, is described in U.S. Pat. No. 5,158,952 according to the following scheme.

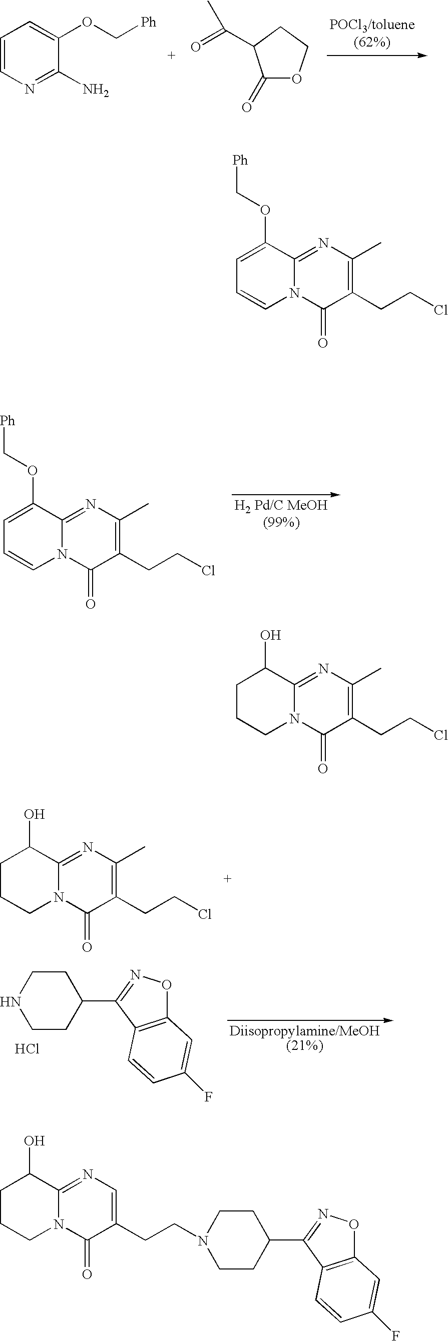

Processes for the synthesis of 3-(2-chloroethyl)-6,7,8,9-tetrahydro-9-hydroxy-2-methyl-4H-pyrrido[1,2-a]pyrimidin-4-one, (‘CMHTP’), an intermediate of Paliperidone, are described in U.S. Pat. No. 5,158,952 and WO 2006/027370.

All the processes described in the prior art are long, and result in low chemical yields, making their application in the industry very hard. There is a need in the art for a new process for preparing Paliperidone and its intermediates.

### SUMMARY OF THE INVENTION

The present invention provides a novel process for the preparation of Paliperidone or CMHTP comprising hydroxylating Risperidone or ClMTTP, i.e., 3-(2-chloroethyl)-6,7,8,9-tetrahydro-2-methyl-4H-pyrrido[1,2-a]-pyrimidin-4-one, respectively, with at least one oxidoreductase enzyme, followed by optionally isolating or purifying the Paliperidone or CMHTP product. The at least one oxidoreductase enzyme used in the process is selected from the group of peroxidases, dioxygenases, monooxygenases and any combination thereof.

### DETAILED DESCRIPTION OF THE INVENTION

The invention provides an economically “green chemistry” process, which avoids the use of potentially harmful reagents.

The enzymatic hydroxylating step of the process for preparing Paliperidone or CMHTP of the present invention preferably is a regioselectively hydroxylating step. As used in the present application, the term “regioselectively hydroxylating”, “regioselectively hydroxylate” or “regioselective hydroxylation” means that the Risperidone or ClMTTP is selectively hydroxylated at the 9-position of the 4H-pyrrido[1,2-a]-pyrimidin-4-one moiety.

As used herein, the term “oxidoreductase enzyme” refers to peroxidases, dioxygenases and monooxygenases. Examples of the monooxygenases are, preferably, cytochrome P450 enzymes, and more preferably CytP450 IID6. Examples of peroxidases are preferably CPO, i.e., chloroperoxidase, and HRP, i.e., horseradish peroxidase.

The term “NADPH” refers to reduced nicotinamide adenine dinucleotide phosphate.

The term “NADP” refers to oxidized nicotinamide adenine dinucleotide phosphate.

The term “NADPH regeneration system” refers to a system of converting NADP to NADPH. An example of the NADPH regeneration system contains glucose-6-phosphate and glucose-6-phosphate dehydrogenase, which converts NADP and glucose-6-phosphate to NADPH and 6-phosphoglucono-6-lactone, respectively. Another example of the NADPH regeneration system contains 6-phosphogluconate and phosphogluconate dehydrogenase, which converts NADP and 6-phosphogluconate to NADPH and ribulose-5-phosphate (Ru5P), respectively. Another example of the NADPH regeneration system contains glucose-6-phosphate, glucose-6-phosphate dehydrogenase, 6-phosphoglucono-6-lactonase and phosphogluconate dehydrogenase, which forms one molecule of Ru5P and two molecules of NADPH from one molecule of glucose-6-phosphate and two molecules of NADP, respectively.

The term “FBIP” means 6-fluoro-3-piperidino-1,2-benisoxazole, which is one of the intermediates of Paliperidone.

As used herein, the term “ferredoxin” means a class of proteins that mediate electron transfer reactions. Preferably, the “ferredoxin” used in some of the processes of the present invention refers to the ferredoxin disclosed in the U.S. patent application publication No. US 2006/0172383, the disclosure of which is incorporated by reference in its entirety.

The Risperidone starting material, as well as the ClMTTP starting material, can be obtained as described in U.S. Pat. No. 4,804,663 and WO 2004/035573.

The process for preparing Paliperidone or CMHTP of the present invention comprises the enzymatical hydroxylation of Risperidone or ClMTTP, respectively, with the at least one oxidoreductase enzyme, followed by optional isolation or purification of the Paliperidone or CMHTP.

Preferably, the enzymatically hydroxylating step regioselectively hydroxylates the Risperidone or ClMTTP.

The obtained Paliperidone or CMHTP can be either enantiomerically enriched, or a racemate. If the product obtained is enantiomerically enriched, it can further be racemized by any conventional methods, such as contacting with an acid.

The at least one oxidoreductase enzyme can be at least one oxidoreductase enzyme of a mammal, preferably human, filamentous fungus, yeast or bacteria. The at least one oxidoreductase enzyme can be obtained, isolated or purified from any host such as a mammal, preferably human, filamentous fungus, yeast or bacteria. The at least one oxidoreductase enzyme can also be at least one oxidoreductase enzyme expressed in a mammalian, filamentous fungal, yeast or bacteria host, wherein the at least one oxidoreductase enzyme gene is taken from an organism such as a human different than the host. The enzymatically regioselective hydroxylation step of the process for preparing Paliperidone or CMHTP of the present invention can be performed with (a) at least one isolated or purified oxidoreductase enzyme, (b) at least one isolated whole cell of a mammal, filamentous fungus, yeast or bacteria naturally containing the at least one oxidoreductase enzyme, or a cellular fraction or cell free extract of the mammal, filamentous fungus, yeast or bacteria having the at least one oxidoreductase enzyme, (c) the whole cell of the mammalian, filamentous fungal, yeast or bacteria host having the at least one oxidoreductase enzyme expressed, (d) a cellular fraction of the mammalian, filamentous fungal, yeast or bacteria host containing the at least one oxidoreductase enzyme expressed in the host, and/or (e) a cell free extract of the mammalian, filamentous fungal, yeast or bacteria host containing the at least one oxidoreductase enzyme expressed. The host can be actinomycetes such as Streptomyces helvaticus, Streptomyces lividans and Penicillium citrinum, ascomycota, Escherichia coli or Bacillus subtilis having both cytochrome P450 and ferredoxin genes expressed as described in U.S. Patent Publication No. 2006/0172383. The host can also be Aspergillus oryzae.

If the enzymatic hydroxylating step of the process for preparing Paliperidone or CMHTP of the invention does not use the isolated whole cell of the mammal, filamentous fungus, yeast or bacteria naturally containing the at least one oxidoreductase enzyme, or the whole cell of the mammalian, filamentous fungal, yeast or bacteria host having the at least one oxidoreductase enzyme expressed, addition of a NADPH regeneration system in the process may be needed if no NADPH regeneration system is originally present. For instance, when the enzymatic hydroxylating step uses a cellular fraction of the mammal, filamentous fungus, yeast or bacteria naturally containing the at least one oxidoreductase enzyme, or cellular fraction of the mammalian, filamentous fungal, yeast or bacteria host containing the at least one oxidoreductase enzyme expressed in the host, a NADPH regeneration system is added in the process if the cellular fraction does not contain any NADPH regeneration system. When the enzymatic hydroxylating step uses a cell free extract of the mammal, filamentous fungus, yeast or bacterial naturally containing the at least one oxidoreductase enzyme, or cell free extract of the mammalian, filamentous fungal, yeast or bacteria host containing the at least one oxidoreductase enzyme expressed, a NADPH regeneration system is added in the process if the cell free extract does not contain any NADPH regeneration system. When the enzymatic hydroxylating step uses at least one isolated or purified oxidoreductase enzyme, a NADPH regeneration system is added in the process.

The optional isolation or purification of the Paliperidone or CMHTP can be conducted by any of the methods known to one skilled in the art. Examples of these methods include precipitation followed by crystallization from an appropriate solvent, flash chromatography and liquid/liquid extraction.

In the enzymatic hydroxylation step of the process for preparing Paliperidone or CMHTP of the present invention, the enzymatic hydroxylation of Risperidone or ClMTTP can be performed with the at least one oxidoreductase enzyme and a source of oxygen, preferably, molecular oxygen. The at least one oxidoreductase enzyme can be in the reduced form.

One of the embodiments of the process for the enzymatic preparation of Paliperidone can be summarized in the following scheme:

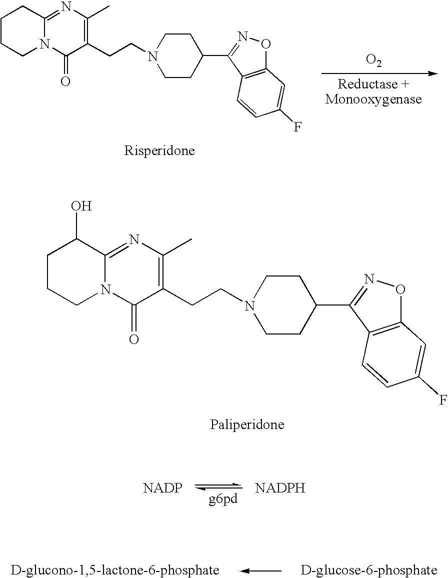

According to the above scheme, in one of the embodiments of the present invention, Papiperidine is produced from risperidone by reduced CytP450 IID6 in the presence of molecular O₂

The electron for the reduction of the cytochrome P450 enzyme such as CytP450 IID6 can be originated from NADPH through an electron transporter chain which includes a reductase such as NADH-cytochrome P450 reductase, wherein NADH represents reduced nicotinamide adenine dinucleotide. The NADPH is regenerated by other enzymes in the NADPH regeneration system. Glucose-6-phosphate-dehydrogenase (g6pd) is an enzyme which can convert glucose-6-phosphate to D-glucono-1,5-lactone 6-phosphate while generating NADPH from NADP.

The present invention also provides a process for the enzymatic preparation of CMHTP summarized in the following scheme:

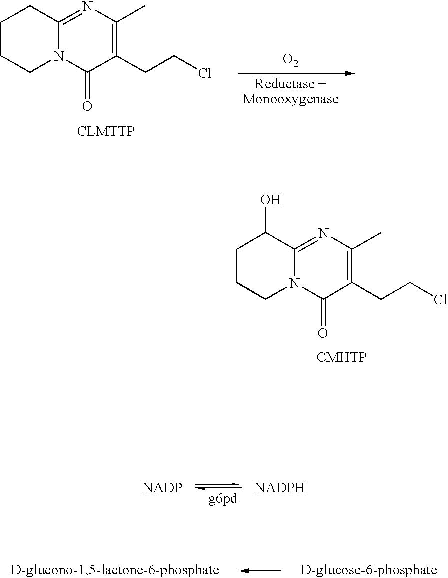

The enzyme used in the process is at least one oxidoreductase enzyme selected from the group of peroxidases, dioxygenases, monooxygenases, preferably cytochrome P450 enzymes, more preferably CytP450 IID6, and any combination thereof.

As described above, the process for the preparation of Paliperidone or CMHTP can be performed by a group of peroxidases, dioxygenases, monooxygenases, preferably cytochrome P450 enzymes, more preferably CytP450 IID6, or any combination thereof. The enzymes can be expressed and obtained from any host such as filamentous fungus, bacteria or yeast. The reaction system can include isolated enzymes or whole-cell catalysis.

If the cytochrome P450 enzymes are used in the process, the electron transfer chain process can be represented as follow:

The processes of the present invention are typically carried out in an aqueous phase. The temperature in which the processes are carried out can be from about 24° C. to about 37° C. One skilled in the art would understand, that the duration in which the processes are carried out can vary, and depends on the specific conditions of the process. Also that higher chemical yields can be obtained by longer incubation. With the description of the invention presented above, the invention is further described by reference to the following examples showing some of the embodiments of the invention. It will be apparent to one skilled in the art that modifications to the examples may be practiced without departing from the scope of the invention.

### EXAMPLES

### Example 1

### Preparation of Paliperidone by Purified Human CytP450 IID6

4 μl of 100 mM Risperidone dissolve in methanol was added to 0.5 nmole CytP450 IID6 expressed and purified from E. coli in the presence of NADPH-P450 reductase and NADPH regeneration system in a 400 μl reaction volume in a 2 ml Eppendorf tube. The reaction was mixed and incubated at 30° C. Risperidone to Paliperidone conversion was assayed at certain time points. After 120 minutes 16% of the initial Risperidone was converted to Paliperidone

### Example 2

### Preparation of Paliperidone by CytP450 of S. helvaticus

Streptomyces helvaticus spore suspension was used to inoculate 50 mL PSI medium (2% glucose, 0.5% soybean meal, 0.5% soy-peptone, 0.01% KH₂PO₄ and 0.1% CaCO₃) and incubated for 2 days with continuous orbital shaking with 300 rpm at 28° C. This preculture was used to inoculate the 50 mL PSF-2 medium (1.8% glucose, 5% soybean meal, 0.4% CSL and 0.3% CaCO₃, pH=7.2) using 10% of inoculum. The culture was grown for 30-40 hours and 1 mL 40 mg/ml risperidone solution (dissolved in methanol) was added to the fermentation culture. After 24 hours of incubation, the fermentation broth was diluted by 4 volumes of methanol and assayed by HPLC. 7.8% of the risperidone added was converted to Paliperidone.

### Example 3

### Preparation of CMHTP by Purified Human CytP450 IID6

4 μl of 100 mM ClMTPP dissolved in methanol was added to 0.5 mmole CytP450 IID6 expressed and purified from E. coli in the presence of NADPH-P450 reductase and NADPH regeneration system in a 400 μl reaction volume in a 2 ml Eppendorf tube. The reaction was mixed and incubated at 30° C. ClMTPP to CMHTP conversion was assayed at certain time points. After 120 minutes, about 3.8% of the initial ClMTPP was converted to CMHTP.

### Example 4

### Preparation of CMHTP by CytP450 of S. helvaticus

S. helvaticus was fermented as described in U.S. patent application US20060172383 except that instead of compactin 1 ml of 40 mg/ml ClMTPP solution (dissolved in methanol) was fed to the fermentation broth. After 24 hours of incubation, the fermentation broth was diluted by 4 volumes of methanol and assayed by HPLC. 5-10% of the fed ClMTPP was converted to CMHTP.

### Example 5

### Preparation of Paliperidone by Aspergillus oryzae

YPD liquid medium (containing: yeast extract 1.0%, soy peptone 2.0%, glucose 2.0%, pH 5.5) was inoculated by A. oryzae vegetative culture. After 24 hours incubation risperidone was fed at 0.5 g/l final concentration. Following 3 days further incubation the fermentation broth was diluted by 4 volumes of methanol and assayed by HPLC. 264 μg/g Paliperidone was detected which is equivalent to about 53% conversion by weight.

### Example 6

### Preparation of CMHTP by Aspergillus oryzae

YPD liquid medium (containing: yeast extract 1.0%, soy peptone 2.0%, glucose 2.0%, pH 5.5) was inoculated by A. oryzae vegetative culture. After 24 hours incubation ClMTPP was fed at 0.5 g/l final concentration. Following 3 days further incubation the fermentation broth was diluted by 4 volumes of methanol and assayed by HPLC. Some of the initial ClMTPP was converted to CMHTP.

Invention claimed:

1. A process for the preparation of a 4H-pyrrido[1,2-a]-pyrimidin-4-one derivative, wherein the 4H-pyrrido[1,2-a]-pyrimidin-4-one derivative is Paliperidone or 3-(2-chloroethyl)-6,7,8,9-tetrahydro-9-hydroxy-2-methyl-4H-pyrrido[1,2-a]-pyrimidin-4-one (‘CMHTP’), said process comprising enzymatically hydroxylating Risperidone or 3-(2-chloroethyl)-6,7,8,9-tetrahydro-2-methyl-4H-pyrrido[1,2-a]-pyrimidin-4-one (‘ClMTTP’), respectively, with at least one oxidoreductase enzyme to form the Paliperidone or CMHTP; and optionally isolating or purifying the Paliperidone or CMHTP, wherein the at least one oxidoreductase enzyme is selected from the group of peroxidases, dioxygenases, monooxygenases and any combination thereof.

2. The process of claim 1, wherein the 4H-pyrrido[1,2-a]-pyrimidin-4-one derivative is Paliperidone.

3. The process of claim 1, wherein the 4H-pyrrido[1,2-a]-pyrimidin-4-one derivative is CMHTP.

4. The process of claim 1, wherein the at least one oxidoreductase enzyme is at least one monooxygenase.

5. The process of claim 4, wherein the at least one oxidoreductase is at least one cytochrome P450 enzyme.

6. The process of claim 5, wherein the at least one oxidoreductase is CytP450 IID6.

7. The process of claim 5, wherein the at least one cytochrome P450 enzyme is obtained from a mammalian, fungal, yeast or bacterial cell.

8. The process of claim 7, wherein the at least one cytochrome P450 enzyme is obtained from a human.

9. The process of claim 5, wherein the hydroxylating step is performed in the presence of an oxygen source.

10. The process of claim 1, wherein the at least one oxidoreductase enzyme is isolated or purified.

11. The process of claim 1, wherein the at least one oxidoreductase enzyme is in (a) at least one isolated whole cell of a mammal, filamentous fungus, yeast or bacteria naturally having the at least one oxidoreductase enzyme, (b) a cellular fraction or cell free extract of a mammal, filamentous fungus, yeast or bacteria naturally having the at least one oxidoreductase enzyme, wherein the cellular fraction or cell free extract contains the at least one oxidoreductase enzyme, (c) at least one whole cell of a mammalian, filamentous fungal, yeast or bacteria host having the at least one oxidoreductase enzyme expressed therein, and/or (d) a cellular fraction or cell free extract of a mammalian, filamentous fungal, yeast or bacteria host having the at least one oxidoreductase enzyme expressed in the host, wherein the cellular fraction or cell free extract contains the at least one oxidoreductase enzyme.

12. The process of claim 11, wherein the at least one oxidoreductase enzyme is in the at least one whole cell of the mammalian, filamentous fungal, yeast or bacteria host having the at least one oxidoreductase enzyme expressed.

13. The process of claim 12, wherein the hydroxylating step is performed with the mammalian, filamentous fungal, yeast or bacteria host grown in culture.

14. The process of claim 11, wherein the at least one oxidoreductase enzyme is in the cellular fraction of the mammalian, filamentous fungal, yeast or bacteria host containing the at least one oxidoreductase enzyme expressed in the host.

15. The process of claim 11, wherein the at least one oxidoreductase enzyme is in the cell free extract of the mammalian, filamentous fungal, yeast or bacteria host containing the at least one oxidoreductase enzyme expressed in the host.

16. The process of any one of claims 12-15, wherein the host is selected from the group consisting of actinomycetes, Streptomyces helvaticus, Streptomyces lividans, Penicillium citrinum, Aspergillus oryzae, other ascomycota, Escherichia coli, and Bacillus subtilis.

17. The process of any one of claims 12-15, wherein the host is selected from the group consisting of Streptomyces helvaticus, Streptomyces lividans, Penicillium citrinum, Escherichia coli, Bacillus subtilis and Aspergillus oryzae.

18. The process of any one of claims 12-15, wherein the host is selected from the group consisting of Streptomyces helvaticus and Aspergillus oryzae.

19. The process of any one of claims 12-15, wherein the host is Streptomyces helvaticus.

20. The process of any one of claims 12-15, wherein the host is Aspergillus oryzae.

21. The process of any one of claims 10, 14 and 15, wherein the hydroxylating step further comprises adding NADH-cytochrome-P450 reductase, NADPH and a NADPH regeneration system.

22. The process of claim 21, further comprises adding ferredoxin in the enzymatically hydroxylating step.

23. The process of claim 1 or 11, wherein the enzymatically hydroxylating step results in regioselective hydroxylation of the Risperidone or ClMTTP.

24. The process of claim 9, wherein the oxygen source is molecular oxygen.

25. The process of claim 7, wherein the at least one cytochrome P450 enzyme is obtained from Streptomyces helvaticus, Streptomyces lividans, Penicillium citrinum, Aspergillus oryzae, Escherichia coli or Bacillus subtilis.

26. The process of claim 1, wherein the at least one oxidoreductase enzyme is at least one peroxidase.

27. The process of claim 26, wherein the at least one peroxidase is selected from the group consisting of chloroperoxidase and horseradish peroxidase.

28. The process of claim 11, wherein the hydroxylating step further comprises adding NADH-cytochrome-P450 reductase, NADPH and a NADPH regeneration system, wherein the at least one oxidoreductase enzyme is (a) isolated or purified, (b) in a cell free extract or cellular fraction of a mammal, filamentous fungus, yeast or bacteria naturally containing the at least one oxidoreductase enzyme without any NADPH regeneration system, or (c) in a cell free extract or cellular fraction of a mammalian, filamentous fungal, yeast or bacteria host having the at least one oxidoreductase enzyme expressed therein without any NADPH regeneration system.
